(19) United States
(12) Patent Application Publication
(10) Pub. No.: US 2023/0355848 A1
(43) Pub. Date: Nov. 9, 2023

(54) BIOCARPET: A LOW PROFILE, MECHANICALLY OPTIMIZED, AND FULLY BIODEGRADABLE ENDOVASCULAR DEVICE FOR TREATMENT OF PERIPHERAL VASCULAR DISEASES

(71) Applicant: UNIVERSITY OF PITTSBURGH - OF THE COMMONWEALTH SYSTEM OF HIGHER EDUCATION, PITTSBURGH, PA (US)

(72) Inventors: Jonathan VANDE GEEST, Gibsonia, PA (US); William R. WAGNER, Gibsonia, PA (US); John J. PACELLA, Bethel Park, PA (US)

(73) Assignee: UNIVERSITY OF PITTSBURGH - OF THE COMMONWEALTH SYSTEM OF HIGHER EDUCATION, PITTSBURGH, PA (US)

(21) Appl. No.: 18/026,041

(22) PCT Filed: Oct. 13, 2021
(86) PCT No.: PCT/US2021/054808

§ 371 Date: Mar. 13, 2023

Related U.S. Application Data
(60) Provisional application No. 63/090,823, filed on Oct. 13, 2020.

(51) Int. Cl. A61L 31/16; A61L 31/14; A61L 31/06; A61F 2/958

(52) U.S. Cl. CPC A61L 31/16; A61L 31/146; A61L 31/06; A61F 2/958; A61L 2300/604

(57) ABSTRACT

The invention relates to biodegradable endovascular devices, and methods for their preparation and use as medical implant devices. The invention includes flexible, drug-eluting, biodegradable endovascular devices that provide optimum treatment of peripheral arterial disease in small arteries and across joints. According to the invention, the biodegradable endovascular medical implant devices include a thermoformed tube or cylinder constructed of a flexible polymeric material, positioned in a vascular region of a patient, wherein the thermoformed tube or cylinder conforms to the geometry or anatomy of the vascular region to treat peripheral arterial disease.

### CROSS-REFERENCE TO RELATED APPLICATIONS

This application claims priority under 35 U.S.C. § 119(e) from U.S. provisional patent application No. 63/090,823, entitled “BIOCARPET: A LOW PROFILE, MECHANICALLY OPTIMIZED, AND FULLY BIODEGRADABLE ENDOVASCULAR DEVICE FOR TREATMENT OF PERIPHERAL VASCULAR DISEASES”, filed on Oct. 13, 2020, the contents of which are incorporated herein by reference.

### GOVERNMENT FUNDING

This invention was made with government support under #HL111990 awarded by the National Institutes of Health (NIH). The government has certain rights in the invention.

### 1. FIELD OF THE INVENTION

The invention relates to flexible, biodegradable endovascular devices as medical implants for the treatment of peripheral vascular diseases.

### 2. BACKGROUND

The introduction of minimally invasive surgical techniques, and the development of various endovascular devices have substantially improved human health care over several decades. Further improvements may be realized by increasing the functionality of these devices and extending the types of procedures where such devices may be employed.

It is believed that improvement in the materials of composition and construction of the endovascular devices will provide increased opportunities for optimizing the benefits derived from the devices. For example, it is desired to utilize different materials to achieve both improved mechanical properties and biodegradability for medical implant devices. The use of degradable components allows a tissue engineering approach to be pursued where no permanent foreign body is left behind in the patient when there is no longer a need for the implanted medical device. Devices or pieces of devices remaining in the patient can potentially pose a risk of infection, fibrosis or abrasion. Constructing devices from biodegradable materials can substantially reduce or eliminate these risks.

There are many known medical conditions and diseases wherein treatment can be improved by the development of improved materials and designs for implantable medical devices. For example, peripheral arterial disease is a common circulatory problem in which narrowed arteries reduce blood flow to your limbs. Symptoms of this disease involving the lower extremities include cramping, pain or tiredness in the leg or hip muscles while walking or climbing stairs. Bending of the knee causes the vessel to bend into complex angles across the joint. This can cause blood flow to decrease even further, causing more pain and severe health issues. Peripheral artery disease is difficult to treat, in particular, in vessels placed where flexion is common, specifically, behind the knee, and the prolonging of the disease may result in infection, tissue death and sometimes amputation.

Peripheral arterial disease has become an increasingly serious public health problem, with 236 million people ages 40 and older being affected world-wide. It also has a large monetary cost, with insurance companies and private payers paying $21 billion annually to cover costs, including medication, physical therapy, and device reintervention. The most common form of treatment has been angioplasty, in which a stent is inserted to increase blood flow. However, it is difficult to treat small arteries with these devices and they suffer from restenosis. The complex anatomies of the smaller vessels prevent the stent from remaining straight, causing kinks that preclude or stop its function. In addition the rigid nature of metallic materials, such as stents, have mechanical disadvantages that can prevent it from properly fitting the vessel. Restenosis related to stents is common—especially in complex bending (cross-joint) applications—leading to an estimated reintervention rate of 70% within two years.

In traditional metallic stents, the design includes a cylinder that is fabricated at a specific (larger) diameter and then compressed to fit around a (much smaller) balloon catheter. This approach limits the size of arteries that can be treated using stents, and also results in non-homeostatic vascular wall stresses in the artery immediately proximal and distal to the lesion, which is known to induce stent restenosis. These issues are drastically exacerbated when treating vascular lesions occurring within complex vascular anatomy and in vessels that span bending joints, as commonly occurs in the knee (e.g., increased stress in the vessel wall during joint flexion after deploying a standard metallic stent in a straight leg configuration). Thus, stents are considered an ineffective treatment option for peripheral arterial disease.

There is a need in the art for the inventive concept that includes a flexible, biodegradable, polymeric, endovascular device (“biocarpet”), which can be drug-eluting, and provides effective treatment of peripheral arterial disease across joints. The biocarpet can be rolled around a balloon catheter, deployed into the complex vascular lesion anatomy, and subsequently thermoformed in situ. This design allows the delivery of extremely thin devices sequentially and thereby, significantly reduces the profile of the devices (during delivery). Further, this design allows the treatment of smaller vascular segments that heretofore were untreatable using standard stent technology. Such treatment is particularly needed in the peripheral vascular beds of the lower leg.

### SUMMARY OF THE INVENTION

An aspect of the invention provides a biodegradable endovascular medical implant device including a thermoformed tube or cylinder positioned in a vascular region of a patient, including a non-porous or intentionally porous, polymeric material in a flexible form, wherein the thermoformed tube or cylinder conforms to the geometry or anatomy of the vascular region, and wherein the thermoformed tube or cylinder is effective to treat peripheral arterial disease.

The polymeric material may include a polymer or blend thereof selected from the group consisting of collagen, gelatin, tropoelastin, polyesters, polyurethanes, polyurethane ureas and, blends and combinations thereof.

In certain embodiments, the flexible form is selected from the group consisting of a cover, sheet, membrane, coating, and matrix.

The geometry or anatomy may be selected from the group consisting of small arteries and cross-joints.

In certain embodiments, the device is effective to treat below-the-knee peripheral arterial disease.

The device may further include a drug eluting mechanism.

Another aspect of the invention provides a method of preparing a flexible, biodegradable endovascular medical implant device. The method includes preparing a flat, flexible material comprised of polymer or blends thereof; obtaining a balloon catheter; wrapping or rolling the flat, flexible material around the balloon catheter with the balloon in its deflated state; inserting the wrapped or rolled flat, flexible material and the underlying balloon catheter in the deflated state into a target vascular region of a patient; subsequently inflating the balloon in the target vascular region; and thermoforming in situ by applying heat to the flat, flexible material to form a tube or cylinder structure that conforms to the geometry or anatomy of the target vascular region.

In certain embodiments, the thermoforming step includes inflating the balloon; increasing the temperature; heating for a period of time; and subsequently deflating the balloon, wherein during this step, the flat, flexible material wrapped or rolled around the balloon catheter that has been inflated, first conforms to the target vascular region anatomy and then after heating, forms a tube or cylinder structure. In certain embodiments, the heating step is performed one or more times with or without a cooling cycle therebetween.

The method can further include a drug-eluting mechanism. In certain embodiments, the drug-eluting mechanism includes attaching a drug directly or indirectly to a surface of the flat, flexible material. The attaching of the drug can be conducted during or subsequent to preparing the flat, flexible material. In certain other embodiments, the drug-eluting mechanism includes encapsulating or embedding a drug into the flat flexible material. The encapsulating or embedding the drug can be conducted during or subsequent to preparing the flat, flexible material. In further embodiments, the drug-eluding mechanism includes a drug stored in a plurality of pores formed in the flat, flexible material.

The drug eluting mechanism provides a controlled and sustained release of one or more pharmaceutical agents.

In still another aspect, the invention provides a method of treating peripheral arterial disease. The method includes obtaining a polymer or blend thereof in a flexible form; applying the flexible form to a balloon catheter to surround or encompass the balloon catheter; deflating the balloon; inserting the flexible form and the underlying deflated balloon catheter into a patient; advancing the flexible form and the underlying deflated balloon catheter into a target vascular region; inflating the balloon; conforming the flexible form to the geometry of the target vascular region; increasing the temperature; and molding the flexible form into a tubular or cylindrical structure that conforms to the geometry of the target vascular region.

### DETAILED DESCRIPTION OF PREFERRED EMBODIMENTS

The invention relates to biodegradable endovascular devices (“biocarpets”), and methods for their preparation and use as medical implant devices. The devices are employed for treating stenotic arterial disease, using a rolled and thermoforming method. The devices conform to the patient's own artery. The devices are fabricated in a manner that their own material stiffness and geometry are optimized to satisfy a targeted vascular wall stress in-vivo.

The invention includes flexible, biodegradable endovascular devices (“biocarpets”), which are optionally drug eluting, that provide effective or optimal treatment of peripheral arterial disease, including in small arteries and across joints. These devices utilize one or more, e.g., a blend, of biocompatible polymers and special in-situ thermoforming techniques, which allow the inventive devices to have structural and mechanical advantages over known stents. Due to the unique thermoformability of the devices they are flexible enough and conformable to be inserted into complex vessel geometries, which is particularly advantageous for treating patients with below-the-knee peripheral arterial disease. Further, due to their biodegradability, the devices are capable of disintegrating once treatment is completed. In addition, the devices have the capacity to deliver a larger amount of drug into the vessel than is capable with conventional drug eluting stents. Thus, these devices have the capability to reduce the risk of restenosis.

The inventive endovascular devices are composed and/or constructed of a flexible, biodegradable material. The material includes synthetic polymers or native biopolymers, or blends thereof. Non-limiting examples of suitable materials for use in the composition and construction of the inventive devices include biocompatible, flexible, biodegradable polymers known in the art, such as, but not limited to, collagen, gelatin, tropoelastin, polyester, polyurethane urea (PUU), polycaprolactone (PCL), poly-L-lactic acid (PLLA), polyglycolic acid (PGA) and, polymer blends and combinations thereof. Further, PUUs possess good biocompatibility with non-toxic degradation products and high elasticity and strength, even in very thin (<1 mm) formats. PUUs include soft segments such as polycaprolactone, polyethylene glycol, polycarbonate, and the like, diisocyanatebutane and chain extender putrescine.

In certain embodiments, the polymers or polymer blends include recombinant human tropoelastin to form a highly deformable polymer or polymer blend for construction of the inventive device.

In certain embodiments, PUU copolymer is prepared by a two-step polymerization process whereby polycaprolactone diol, 1,4-diisocyanatobutane, and diamine are combined in a 1:2:1 molar ratio. In the first step, a pre-polymer is formed by reacting polycaprolactone diol with 1,4-diisocyanatobutane. In the second step, the pre-polymer is reacted with diamine to extend the chain and form the final polymer.

The degradation profiles and mechanical properties of the polymers or polymer blends can be tailored or pre-selected by changing or varying the molecular weight and the composition of the soft segments. In certain embodiments, the endovascular devices according to the invention are fully (i.e., 100%) degradable.

A thermoplastic elastomer is easily processed into various different shapes and forms. Of specific interest to tissue engineering applications, scaffolds (being non-porous or intentionally porous) are made from polyurethanes using fabricating processes, such as, thermally induced phase separation, salt leaching, and electrospinning. The resulting polymeric material is in a flexible form, such that it is conformable to various complex, arterial geometries such as small arteries and cross-joints. In certain embodiments, the polymeric material is in a flat, flexible form, such as but not limited to, a flat, flexible sheet, cover, membrane, matrix or coating.

In certain embodiments, the endovascular devices according to the invention are composed and/or constructed of a flat, flexible polymeric sheet comprising one or more of the aforementioned synthetic and native biopolymers.

The flexibility of the polymeric materials used for the composition and/or construction of the endovascular devices allows the material, e.g., in the form of a flat, flexible polymer sheet, to be wrapped or rolled onto and/or around the outside surface of a balloon catheter to surround or encompass the balloon in its deflated state, e.g., the balloon catheter, e.g., deflated balloon, is positioned within an open (interior) space formed by the wrapped or rolled material.

In certain embodiments, the mechanical stiffness and two-dimensional (2D) geometry of the polymeric material (before being wrapped or rolled onto and/or around the deflated balloon catheter) is tuned such that the resulting endovascular device demonstrates the following properties and characteristics:
a) Conforms to the local complex geometry of a host artery (even when the host artery is located in a joint that is bent) when thermoformed;
b) Minimizes vascular wall stress post deployment;
c) Delivers a heretofor unachievable amount of anti-restenosis drug;
d) Minimizes surface area to reduce thrombogenic potential; and
e) Promotes device host integration and endothelialization.

The wrapped or rolled polymeric material containing the balloon catheter with the balloon deflated is then inserted into a patient, and advanced as-is into a target vascular region for implantation. In certain embodiments, the target vascular region is a complex vascular lesion anatomy such as a diseased peripheral vascular segment or host artery. Once inserted in the target vascular region, the balloon is inflated and a thermoforming process is conducted in-situ to locally deploy the endovascular device. Inflating the balloon allows the wrapped or rolled polymeric material, due to its flexibility, to expand and conform to the geometry or anatomy of the target vascular region. After inflating the balloon, the temperature is increased and heat is applied to the polymeric material. The heat is supplied by using one or more conventional heating elements known in the art. Following the heating step, the balloon is deflated. Typically, the heat is applied for a period of seconds. In certain embodiments, the heating step is repeated one or more times. In certain embodiments, a cooling cycle is implemented between each of the subsequent heating steps. The period of time for heating and the number of heating steps varies. The heating is conducted such that the wrapped or rolled polymeric material is transformed, re-configured, or molded. During and following the thermoforming process, the polymeric material, e.g., flat, flexible sheet, is formed into a tubular or cylindrical shaped structure or article, e.g., a tube or cylinder, that conforms to the geometry or anatomy of the target vascular region. Subsequently, the balloon catheter is removed and the tube or cylinder structure remains in place in the target vascular region. In certain embodiments, the thermoformed device is not in contact with the walls of the target vascular region, e.g., host artery.

The thermoforming process in-situ, which transforms the flat, flexible polymer sheet (e.g., wrapped or rolled around the deflated balloon catheter) into a tube or cylinder, allows for the delivery of extremely thin devices sequentially and thereby, significantly reduces the profile of the devices (during delivery). In certain embodiments, the inventive device is thermoformed onto itself, allowing any thickness of the device to result in-vivo post thermoforming. In certain embodiments, by thermoforming the inventive device onto itself, the resultant thermoformed structure or article includes multiple, e.g., sequential, sheets or layers in a stacked arrangement or configuration.

The low profile and thermoformability of the device allows it to be delivered into smaller diameter peripheral vessels using a sequential low profile approach, thus allowing the treatment of peripheral vascular disease that is not currently achievable with purely metallic devices, e.g., stents. These design features are capable of reducing the restenosis rate that is commonplace in the endovascular treatment of peripheral arterial disease, and thereby eliminating the need for secondary interventions that occur within one year in approximately 25 to 50% of peripheral arterial disease (PAD) patients.

Design optimization of the device involves the use of both a computational approach and an experimental approach. The inventive device is fabricated employing either subtractive or additive manufacturing using either single or two photon laser cutting or polymerization, respectively. For the prior, this will be done on either solvent casted, electrospun, injected molded or similar flat sheets. In certain embodiments, the device fabrication method is a two-photon laser cutting fabrication technique that allows programmatic control over the pre-rolled 2D biocarpet geometry.

The conventional approach for treatment of peripheral arterial disease involves metallic stents, which include a cylinder that is fabricated at a specific (larger) diameter and then compressed to fit around a (much smaller) balloon catheter. This approach limits the size of arteries that can be treated using stents, and also results in non-homeostatic vascular wall stresses in the artery immediately proximal and distal to the lesion, which is known to induce stent restenosis. These issues are drastically exacerbated when treating vascular lesions occurring within complex vascular anatomy and in vessels that span bending joints, as commonly occurs in the knee (e.g., increased stress in the vessel wall during joint flexion after deploying a standard metallic in a straight leg configuration). Whereas, the inventive device is composed of a flexible, biodegradable polymer form, e.g., sheet, that is wrapped or rolled onto and/or around a deflated balloon catheter, deployed into the complex vascular lesion anatomy, and subsequently thermoformed in situ to conform to the geometry or anatomy of the target vascular anatomy.

Thus, in accordance with certain embodiments of the invention, a flexible polymer sheet is employed to surround or encase a deflated balloon catheter that is then inserted into a vascular lesion anatomy, and subsequently deployed by thermoforming in-situ. The in-situ thermoforming process involves the inflation of the underlying balloon, and then heating of the inventive device (e.g., flexible polymer sheet) for repeated short time periods, e.g., seconds. As a result of in-situ thermoforming, the inventive device becomes a tube or cylinder article that conforms to the geometry of the host artery. Thus, thermoformability allows the inventive device to be molded into the shape or geometry of the host artery. The tubular or cylindrical device provides equivalent radial resistance to a stent while simultaneously providing a homoeostatic distribution of vascular wall stress within the artery both proximal and distal to the lesion.

Complete tunability of the device's mechanical properties and 2D pre-rolled geometry further allow minimization of vascular wall stress across the entire treated lesion. Moreover, the thermoforming approach allows the potential to deploy the device in bent joint configuration. This itself reduces the wall stress in treated arteries as compared to traditional metallic stents delivered in straight joint configurations.

As the thermoforming process described above indicates, the inventive device is thermoformed to itself. As a result, if needed, extremely thin devices are delivered sequentially, thereby significantly reducing the profile of the device (during delivery). This allows the device to treat smaller vascular segments heretofor untreatable using standard stent technology. This is especially needed in the peripheral vascular beds of the lower leg.

There are conventional drug-eluting stents that are known to treat restenosis, as it is the primary failure mode of the endovascular treatment of peripheral arterial disease. However, there are inherent limitations in the volume of anti-restenotic drug that can be delivered when the drug is incorporated within a coating on a non-biodegradable metallic stent. In contrast, the inventive device is fully biodegradable and therefore, allows the delivery of significantly larger amounts of anti-restenotic and/or antithrombotic drug as the device degrades into the patient body over a period of time. As an added benefit, this increased delivery volume allows for more efficient and improved approaches in controlled temporal delivery of the drug. For example, the ability to include and elute both acute and chronic therapeutic drugs within the same device. The various mechanisms known in the art for eluting drugs from a polymeric implant device are applicable to the inventive device. These methods include, but are not limited to, attaching a drug directly or indirectly to the polymeric material surface of the device, and encapsulating or embedding a drug into the polymeric material, e.g., matrix. The attaching and encapsulating or embedding of the drug to or into the polymer material is performed either during or subsequent to preparation of the polymer. In certain embodiments, the polymeric material contains a multiple or a plurality of pores, intentionally, for storage of a drug to be eluted from the device. The drug eluting mechanism of the inventive device incudes the controlled and sustained release of various pharmaceutical agents, which are known in the art.

Thus, the inventive device includes one or more of the following advantages as compared to conventional metallic stents:
a) Integration of the device into the host vasculature as well as the mechanical benefit of having a highly deformable polymer exist in the device by the inclusion of recombinant human tropoelastin; thermoformability allows the device to be molded into the shape of the host artery;
b) Optimized mechanically and structurally for small artery and bending applications;
c) Elution of anti-restenosis medications;
d) Utilization of biopolymers to encourage a desired host vascular remodeling and integration; and
e) 100% biodegradable to minimize long term complications.

Whereas particular embodiments of the invention have been described herein for purposes of illustration, it will be evident to those skilled in the art that numerous variations of the details may be made without departing from the invention as set forth in the appended claims.

We claim:

1. A biodegradable endovascular medical implant device, comprising:
a thermoformed tube or cylinder positioned in a vascular region of a patient, comprising:
a non-porous or intentionally porous, polymeric material in a flexible form,
wherein the thermoformed tube or cylinder conforms to the geometry or anatomy of the vascular region, and
wherein the thermoformed tube or cylinder is effective to treat peripheral arterial disease.

2. The device of claim 1, wherein the polymeric material comprises a polymer or blend thereof selected from the group consisting of collagen, gelatin, tropoelastin, polyesters, polyurethanes, polyurethane ureas and, blends and combinations thereof.

3. The device of claim 1, wherein the flexible form is selected from the group consisting of a cover, sheet, membrane, coating, and matrix.

4. The device of claim 1, wherein the geometry or anatomy is selected from the group consisting of small arteries and cross-joints.

5. The device of claim 1, wherein said device is effective to treat below-the-knee peripheral arterial disease.

6. The device of claim 1, wherein said device further comprises a drug eluting mechanism.

7. A method of preparing a flexible, biodegradable endovascular medical implant device, comprising:
preparing a flat, flexible material comprised of polymer or blends thereof;
obtaining a balloon catheter;
wrapping or rolling the flat, flexible material around the balloon catheter with the balloon in its deflated state;
inserting the wrapped or rolled flat, flexible material and the underlying balloon catheter in the deflated state into a target vascular region of a patient;
subsequently inflating the balloon in the target vascular region; and
thermoforming in situ by applying heat to the flat, flexible material to form a tube or cylinder structure that conforms to the geometry or anatomy of the target vascular region.

8. The method of claim 7, wherein the thermoforming step comprises:
inflating the balloon;
increasing the temperature;
heating for a period of time; and
subsequently deflating the balloon,
wherein, during this step, the flat, flexible material wrapped or rolled around the balloon catheter that has been inflated, first conforms to the target vascular region anatomy and then after heating, forms a tube or cylinder structure.

9. The method of claim 8, wherein the heating step is performed one or more times with or without a cooling cycle therebetween.

10. The method of claim 7, further comprising a drug-eluting mechanism.

11. The method of claim 10, comprising attaching a drug directly or indirectly to a surface of the flat, flexible material.

12. The method of claim 10, comprising encapsulating or embedding a drug into the flat flexible material.

13. The method of claim 11, wherein the attaching the drug is conducted during or subsequent to preparing the flat, flexible material.

14. The method of claim 12, wherein the encapsulating or embedding the drug is conducted during or subsequent to preparing the flat, flexible material.

15. The method of claim 10, comprising a drug stored in a plurality of pores formed in the flat, flexible material.

16. The method of claim 10, wherein the drug eluting mechanism provides a controlled and sustained release of one or more pharmaceutical agents.

17. A method of treating peripheral arterial disease, comprising:
obtaining a polymer or blend thereof in a flexible form;
applying the flexible form to a balloon catheter to surround or encompass the balloon catheter;
deflating the balloon;
inserting the flexible form and the underlying deflated balloon catheter into a patient;
advancing the flexible form and the underlying deflated balloon catheter into a target vascular region;
inflating the balloon;
conforming the flexible form to the geometry of the target vascular region;
increasing the temperature; and
molding the flexible form into a tubular or cylindrical structure that conforms to the geometry of the target vascular region.
